(19) United States
(12) Patent Application Publication
(10) Pub. No.: US 2016/0058839 A1
(43) Pub. Date: Mar. 3, 2016

(54) LEPTIN FOR TREATING SYSTEMIC INFLAMMATORY RESPONSE SYNDROME

(71) Applicant: UNIVERSITY OF VERMONT AND STATE AGRICULTURAL COLLEGE, Burlington, VT (US)

(72) Inventor: Benjamin T. SURATT, Essex, VT (US)

(73) Assignee: UNIVERSITY OF VERMONT AND STATE AGRICULTURAL COLLEGE, Burlington, VT (US)

(21) Appl. No.: 14/888,128

(22) PCT Filed: May 2, 2014
(86) PCT No.: PCT/US14/36485

§ 371 Date: Oct. 30, 2015

Related U.S. Application Data
(60) Provisional application No. 61/818,701, filed on May 2, 2013.

(51) Int. Cl. A61K 38/22; A61K 47/48; A61K 9/00

(52) U.S. Cl. CPC A61K 38/2264; A61K 9/007; A61K 47/48215

(57) ABSTRACT

Provided are methods and compositions for reducing the severity of systemic inflammatory response syndrome (SIRS). The method comprises administering to an individual who has been diagnosed with SIRS, a composition comprising leptin.

### CROSS REFERENCE TO RELATED APPLICATIONS

This application claims priority to U.S. Provisional application no. 61/818,701, filed on May 2, 2013, the disclosure of which is incorporated herein by reference.

### STATEMENT REGARDING FEDERAL FUNDING

This invention was made with government support under grant number HL084200 from the National Institutes of Health. The government has certain rights in the invention.

### BACKGROUND OF THE INVENTION

Systemic Inflammatory Response Syndrome (SIRS) accompanies and is the underpinning of most critical illness associated with organ failure, and, particularly in the setting of the associated conditions of sepsis and the acute respiratory distress syndrome (ARDS), accounts for the majority of ICU deaths (>250,000/year) and ICU expenditures (>$25 billion/year) in the US alone. Despite much effort, no specific therapy has proven effective for this condition.

Although the immune system is critical in the defense against infections, SIRS represents an inflammatory ‘over-response’ to injury or infection that is severe and sustained, leading to diffuse tissue injury, multi-organ dysfunction, and ultimately death in >50% of those severely affected. Although many supportive therapies have been developed for patients with SIRS, no specific therapy has proven to be both effective and free of life-threatening side effects. Recent approaches using recombinant biological agents have been transiently adopted, including both activated protein C (Xigris, Eli Lilly) and insulin infusion (targeting tight blood glucose control). Yet, both yielded substantial iatrogenic morbidity and mortality (hemorrhage and hypoglycemia, respectively) and have since been abandoned.

### SUMMARY OF THE INVENTION

This disclosure provides methods and compositions for reducing the inflammatory response generally referred to as SIRS, and reducing the associated morbidity and mortality. The disclosure is based on the findings that in patients exhibiting SIRS, elevated blood leptin levels were found to be correlated with attenuated inflammation.

In one embodiment, the present disclosure provides a method for reducing the severity of SIRS with or without an accompanying organ failure comprising administering to an individual who has been diagnosed with SIRS and optionally additionally with organ failure, a composition comprising leptin. In one embodiment, neutrophil proinflammatory function (which is central to the pathogenesis of SIRS) is attenuated and neutrophil levels (such as in bronchoalveolar lavage) are reduced, by the administration of leptin. The composition comprising leptin may be administered by any suitable route including parenteral, oral, pulmonary, nasal, and transdermal.

### BRIEF DESCRIPTION OF THE DRAWINGS

FIG. 1. Lipopolysaccharide (LPS)-induced airspace neutrophilia decreases with increasing plasma leptin levels. Bronchoalveolar lavage neutrophil levels graphed against plasma leptin levels from mice with diet-induced obesity (10% vs 60% fat diet for 20 wks) 24 h after nebulized LPS exposure. Leptin measured by plasma ELISA at time of BAL. n=14 mice/group.

FIG. 2. Induced hyperleptinemia (HL) attenuates inflammatory response in LPS-induced SIRS. BAL (A) neutrophil levels and (B) inflammatory cytokine levels from HL and control mice 24 h after nebulized LPS exposure. n=8 mice/group,*p<0.05.

FIG. 3. Hyperleptinemia (HL) induced immediately following injury attenuates inflammatory response in LPS-induced SIRS. BAL neutrophil levels from mice that received leptin (40 μg/kg IP) immediately after injury with nebulized LPS and control (IP PBS) mice 24 h after LPS exposure. n=4 mice/group,*p<0.05.

FIG. 4. Induced hyperleptinemia impairs neutrophil chemotaxis and survival. Mature bone marrow neutrophils were isolated from uninjured HL and control mice and examined in vitro using (A) modified a Boyden chemotaxis chamber (Molecular Probes) with KC and fMLP, and (B) 12 h culture with Fas ligand (200 ng/mL)+/−G-CSF (25 ng/mL). *p<0.05.

FIG. 5. Induced hyperleptinemia reduces neutrophil transcription of the inflammatory cytokine KC. Neutrophils were isolated from HL and control mice and were stimulated in vitro with LPS for 4 h before mRNA was isolated and evaluated by qPCR and normalized to unstimulated buffer-treated controls. *p<0.05.

FIG. 6. Neutrophils phosphorylate p38 and GSK3 in response to leptin. Mature bone marrow neutrophils were isolated from wild type mice and examined using immunoblotting of whole cell lysates for phospho-p38 and GSKα/β after in vitro exposure to IL-6 (20 ng/mL), leptin (25 ng/mL) or G-CSF (25 ng/mL). GSK image has been altered to fit.

FIG. 7. Hyperleptinemia impairs neutrophil signaling response to LPS. Mature bone marrow neutrophils were isolated from HL and control mice and examined using immunoblotting of whole cell lysates for phospho-p38 after in vitro exposure to LPS (100 ng/mL). Ratios of phospho-p38 to total p38 is presented as calculated by blot densitometry.

FIG. 8. Induced hyperleptinemia impairs neutrophil signaling response to G-CSF and IL-6. Mature bone marrow neutrophils were isolated from HL and control mice and examined using immunoblotting of whole cell lysates for phospho-STAT3 and p38 after in vitro exposure to (A) G-CSF (25 ng/mL) or (B) IL-6 (20 ng /mL).

### DETAILED DESCRIPTION OF THE INVENTION

Leptin was first discovered in the obese mouse as a serum factor that decreased food intake and body weight. Because of these initial observations, much of the earlier therapeutic attempt using this hormone has been in the treatment of obesity.

We have observed that elevated blood levels of the protein leptin (“hyperleptinemia”) are associated with attenuated inflammation in critically ill patients. We also observed that leptin has protective effects when injected during systemic inflammatory response syndrome (SIRS) in animal models. The present invention provides compositions and methods for reducing the severity of SIRS and the associated morbidity and mortality.

SIRS typically follows generalized infection, trauma, thermal injury, or sterile inflammatory processes such as acute pancreatitis. SIRS is a clinical diagnosis and is considered to be present when patients have more than one of the following clinical findings: (Crit Care Med 20:864-874, 1992):
Body temperature higher than 38° C. or lower than 36° C.
Heart rate higher than 90/min
Hyperventilation evidenced by respiratory rate higher than 20/min or PaCO₂ lower than 32 mmHg
White blood cell count higher than 12,000 cells/μl or lower than 4,000/μl

For the present invention, the method is useful for patients diagnosed with SIRS and who also have clinically diagnosed organ failure. Organ failure refers to failure of one or more organs. For example, the failure may involve respiratory failure—in which case it is known as ARDS, cardiovascular failure (shock), renal failure or neurologic failure (comatose). Organ failure is typically diagnosed by a clinician and means a state where the organ or system in question has failed to the degree where therapeutic support is required to maintain homeostasis and survival (e.g. mechanical ventilation for respiratory failure, vasopressors for shock). Clinicians are well versed with diagnosis of SIRS and organ failure.

In one embodiment, this invention provides methods and compositions for reducing the severity of or treating Acute Respiratory Distress Syndrome (ARDS), one form of SIRS-associated organ failure.

In one embodiment, this invention provides a method of reducing the activity of neutrophils after acute inflammatory injury. One measure of activity level of neutrophils is the measurement of bronchoalveolar lavage (BAL) neutrophils. Other measures include impairment in neutrophil chemotaxis or inflammatory cytokine transcription, and increased apoptosis of neutrophils.

The method comprises administering to an individual in need of treatment a therapeutically effective amount of leptin (including a salt of leptin), leptin analog, leptin derivative or combinations thereof in pharmaceutically acceptable carrier(s). In one embodiment, the leptin is pegylated or methionylated (Sigma Aldrich). Pegylation of polypeptides is known in the art. The leptin may be recombinant human leptin (PEG-OB, Hoffman La Roche) or recombinant methionyl human leptin (Amgen). Any suitable PEG can be used to pegylate leptin. For example, PEG of from 5 to 100 KDa can be used.

In one embodiment, an approximately 40 KDa PEG is used to pegylate leptin. In one embodiment, the leptin is human leptin, meaning that it is isolated from a human source or has the same sequence as the human leptin. In one embodiment, it is a 167 amino acid protein whose sequence is under GenBank Accession no. AAH69452.1 GI: 46854679, Jun. 9, 2008 entry.

Leptin may be isolated, or produced synthetically or recombinantly. Pharmaceutical compositions of leptin may comprise effective amounts of leptin, leptin analog or leptin derivative together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used, and this may have the effect of promoting sustained duration in the circulation. Agents like oleic acid, linoleic acid and linolenic acid may be added to aid delivery. Further, the formulations may be prepared as controlled release formulations. See, e.g., Remington's Pharmaceutical Sciences, 18th Ed. (1990, Mack Publishing Co., Easton, Pa. 18042). The compositions may be prepared in liquid form, or may be in dried powder, such as lyophilized form, or as aerosolized form.

Leptin formulations may be administered via injection (i.v., i.p. s.q. and the like), oral, pulmonary, nasal, transdermal or other forms of administration. In one embodiment, the leptin formulation may be delivered directly to the respiratory system via a formulation that is suitable for inhalation. For example, the compositions of the invention may be used in the form of drops or sprays (e.g., a nasal spray, aerosol spray, or pump spray) or other vehicles for nasal administration (intranasal delivery). Aerosol spray preparations can be contained in a pressurized container with a suitable propellant such as a hydrocarbon propellant. Pump spray dispensers can dispense a metered dose or a dose having a specific particle or droplet size. Any dispensing device can be arranged to dispense only a single dose, or multiple doses. The formulations may be provided in the form of a dispersible dry powder which can be then prepared for delivery by inhalation. In one embodiment, it is continuously administered to a patient such as through intravenous route or other suitable routes.

The dosage and number of administrations are dependent on the severity of the condition as well as individual patients. Adjusting the dosage and frequency of administration is well within the purview of a skilled physician.

In one embodiment, a suitable dosage is such that it will provide a plasma (or serum leptin level of at least 25 ng/ml. In one embodiment, the serum or plasma leptin levels are 25 to 100 ng/ml and all integers therebetween. In another embodiment, the serum or plasma leptin levels are 30 to 50 or 40 to 50 ng/ml. In another the serum or plasma leptin levels are from 20 to 200 ng/ml and all integers and ranges therebetween

In one embodiment, a suitable dosage is a regimen such that the symptoms leading to the diagnosis of SIRS plus organ failure are reduced. The administration of the leptin can be monitored clinically and administration reduced or stopped when the clinical manifestation of SIRS and organ failure are no longer detectable.

In one embodiment, prior to administration of leptin, serum concentration of leptin may be determined. Such determination can be made by commercially available immunoassays. The dose of leptin administered may be adjusted so as to achieve a serum leptin concentration of 25 to 100 ng/ml, or 20 to 200 ng/ml including all integers and ranges therebetween.

In one embodiment, a suitable dosage of leptin is from 0.1 to 5.0 mg/kg body weight, including all values to the tenth decimal place and all ranges therebetween. In different embodiments, the suitable dosage is 0.1, 0.2, 0.3, 0.4, 0.5, 1.0, 1.5, 2.0, 2.5, 3.0, 3.5, 4.0, 4.5, and 5.0 mg/kg body weight. In some embodiments, suitable dosages include 0.288, 0.576, 1.152, and 2.304 mg/kg body weight. These correspond to 100, 200, 400 and 800 ng/kg/min for infusion purposes.

In one embodiment, the leptin administration can be accompanied by the administration of antimicrobial agents such as antibiotics or antifungal agents. The leptin formulation can be administered before, during or after the administration of the antimicrobial agents.

In one embodiment, this invention provides a method for reducing the symptoms of SIRS and failure of at least one organ, comprising the steps of identifying a patient as being affected by SIRS and organ failure and administering a suitable dose of leptin such that the patient is no longer considered to be have SIRS and organ failure.

In one embodiment, the leptin is administered continuously (such as intravenously) until the diagnosis of SIRS and organ failure can no longer be made.

In one embodiment, the organ failure is a neurologic failure, respiratory failure, failure of the cardiovascular system, liver, hematologic/coagulation, or kidney failure.

In one embodiment, the present disclosure provides a composition comprising leptin for use in reducing the symptoms associated with SIRS, with or without organ failure. In one embodiment, the present disclosure provides leptin for treatment of SIRS and associated organ failure.

The invention is further described through the following examples which are to be considered as illustrative and are not intended to be limiting.

### Example 1

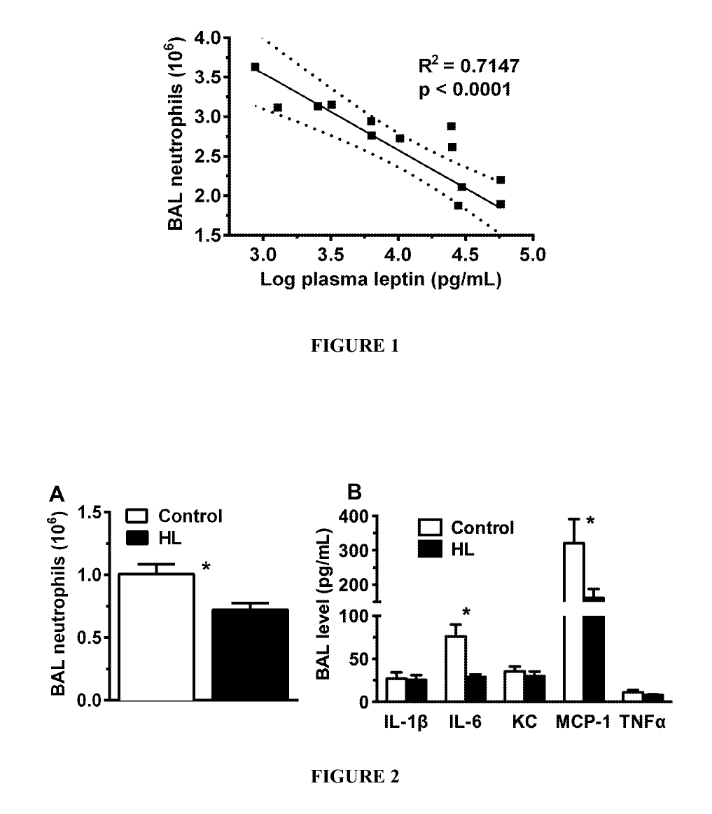

We examined the association between plasma leptin and inflammatory response in our obese mouse models of SIRS. In diet-induced obese and lean mice, hyperleptinemia was strongly associated with attenuated BAL neutrophilia (FIG. 1). Work in our lab has shown that body mass per se is not associated with reduction in inflammation and tissue injury in multivariate analysis (p=0.796), yet systemic leptin levels are significantly associated. (p=0.021).

Examination of plasma leptin levels in a small cohort of SIRS patients (n=388) revealed an inverse relationship between leptin and the inflammatory cytokine IL-6 which is critical to the pathogenesis of SIRS and organ failure. This correlation was independent of body mass index, gender, diabetic status, disease severity, and inciting injury (p=0.047). Thus, elevation of plasma leptin levels appears to be associated with reduced inflammation and tissue injury in SIRS.

### Example 2

An animal model for acute lung injury was used. The model was created by exposing mice to aerosolized E. coli 0111:B4 lipopolysaccharide (LPS, Sigma, St. Louis, Mo.). To test whether protection is conferrable through the therapeutic induction of hyperleptinemia, we administered (daily i.p.) lean mice with recombinant pegylated murine leptin (obtained from Capital Biosciences, Rockville, Md.). Animals were administered repeated daily pegylated recombinant leptin injection (40 μg/kg IP daily). This regimen yielded hyperleptinemia (HL) compared to controls (22.6±3.3 ng/mL vs 1.3±0.7 ng/mL) without weight loss. Although lower than levels seen in mice with a relevant hyperleptinemic condition such as diet-induced obesity (38.2±4.8 ng/mL), we found that leptin treatment significantly attenuated the inflammatory response to this injury (FIG. 2; *=p<0.05). FIG. 2 shows that hyperleptinemia attenuates lung inflammation following LPS exposure. BAL neutrophil levels and inflammatory cytokine levels 24 h after nebulized LPS exposure from mice injected with 40 ug/kg PEG-leptin IP/d x14d (HL) compared to PBS controls. n=8 mice/group, *p=0.01.

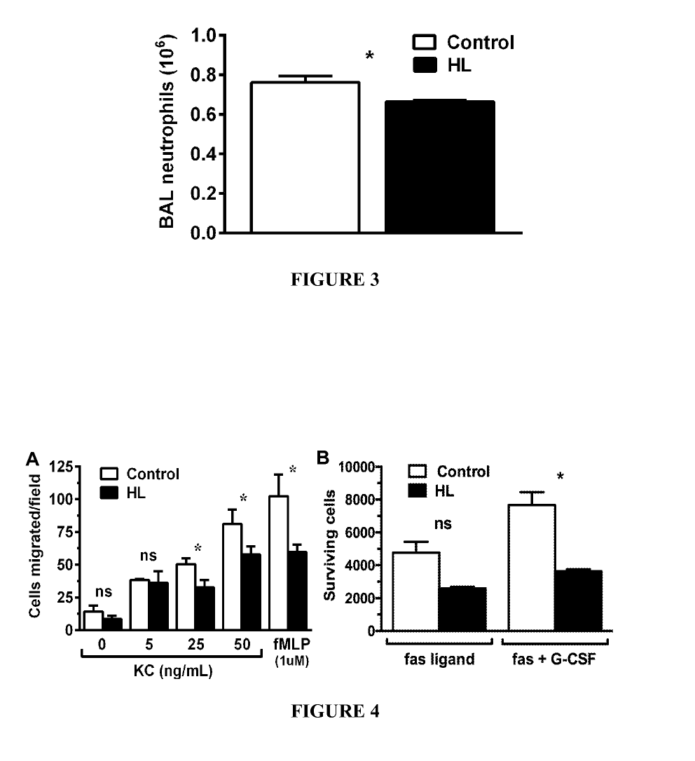

Additional work (FIG. 3) has shown that dosing (40 μg/kg IP) at the time of injury can induce this same effect

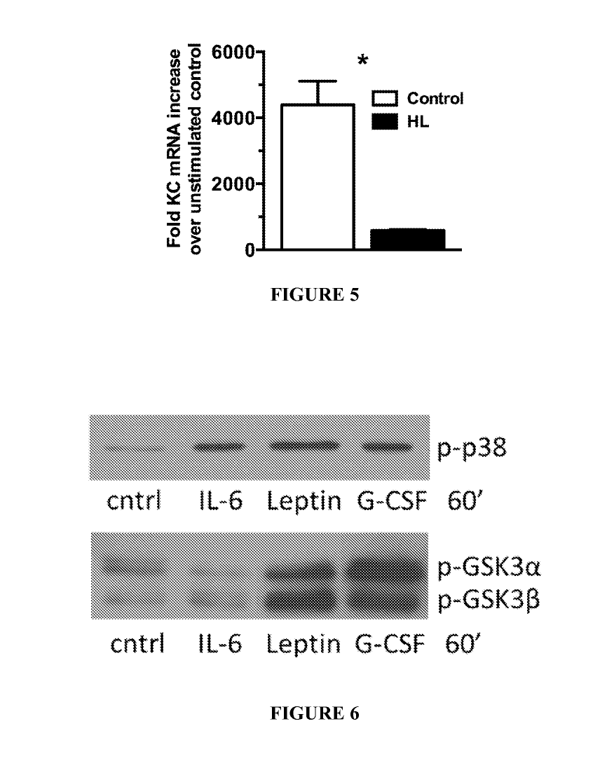

In vitro examination of neutrophils isolated from uninjured HL mice revealed that these cells show attenuated activation and function following exposure to chemokines and bacterial products such as fMLP (formyl-Methionyl-Leucyl-Phenylalanine, a bacterial peptide chemoattractant) and LPS. Neutrophils from HL mice had impaired chemotaxis response to both the chemokine KC (the mouse homologue of human IL-8), and fMLP (FIG. 4a; *=p<0.05, ns=not significant). Neutrophil cell death (and apoptosis by dUTP nick end labeling (TUNEL) staining) was accelerated in HL neutrophils, as well (FIG. 4b). Induction of neutrophil mRNA transcription for KC (a critical pro-inflammatory signal) in response to LPS was also impaired in HL neutrophils (FIG. 5; *=p<0.05). Taken together, these findings indicate that the hyperleptinemic state has significant anti-inflammatory effects at least in part due to inhibition of neutrophil activation, functional response, and survival. Such defects also appear to extend to the function of macrophages, a critical source of inflammatory cytokines, as cytokine levels found in the lungs of injured HL mice appear to be significantly lower than in control mice 24 h after LPS injury (FIG. 2b).

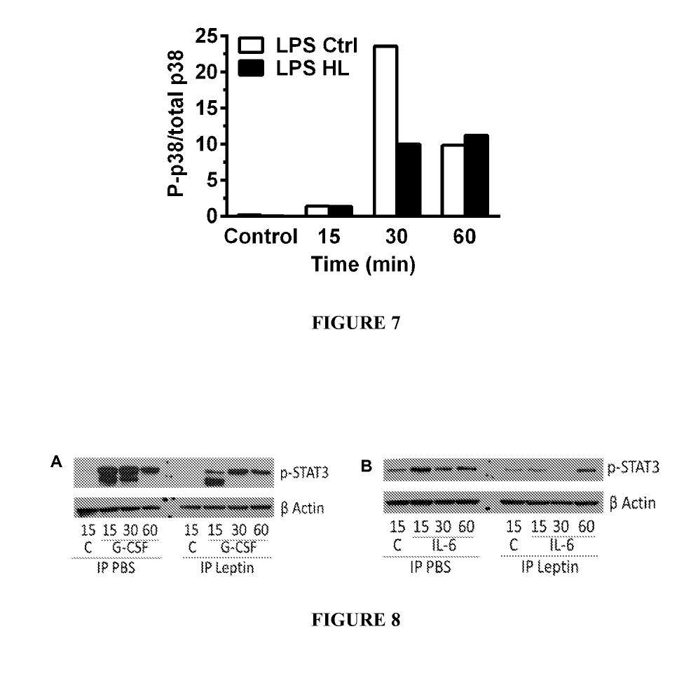

Studies in our lab examining the effects of high dose leptin on leukocyte signaling suggest that this molecule induces prolonged activation of the MAPK pathway (as indicated by p38 phosphorylation in normal neutrophils, FIG. 6a) and the IRS/PI₃K pathway (as indicated by the downstream phosphorylation of GSK3, FIG. 6b), thereby inhibiting cell activation following stimuli. Thus, HL conditions in vivo are found to inhibit both p38 MAPK signaling (critical for inflammatory cell activation and survival, FIG. 7) and STAT3 signaling in response to cytokines prolonging cell survival (IL-6 and G-CSF, FIG. 8) in neutrophils.

The observed ameliorative effects of hyperleptinemia on SIRS accompanied by insignificant toxicity indicate the usefulness of leptin for therapy for this disease.

While the invention has been described through specific embodiments, those skilled in the art will recognize that routine modifications to the disclosure can be made and such modifications are intend to be within the scope of this disclosure.

1. A method of reducing the severity of systemic inflammatory response syndrome (SIRS) comprising administering to an individual in need of treatment, an effective dose of leptin in a pharmaceutically acceptable carrier, wherein said administration reduces the severity of SIRS.

2. The method of claim 1, wherein the individual in need of treatment displays more than one of the following indicators: body temperature higher than 38° C. or lower than 36° C.; heart rate higher than 90/min; hyperventilation evidenced by respiratory rate higher than 20/min or PACO₂ lower than 32 mmHg; and white blood cell count higher than 12,000 cells/μl or lower than 4000/μl.

3. The method of claim 2, further comprising the step of measuring the reduction in the severity of SIRS by measuring said indicators.

4. The method of claim 3, wherein reduction in the severity of SIRS is measured by measuring the activity of neutrophils.

5. The method of claim 4, wherein the neutrophils are bronchoalveolar lavage neutrophils.

6. The method of claim 1, wherein the individual also displays failure of at least one organ or system.

7. The method of claim 6, wherein the failure of an organ or system is one or more of the following neurologic failure, respiratory failure, cardiovascular failure, renal failure, liver failure, and kidney failure.

8. The method of claim 1, wherein leptin is administered by a route selected from the group consisting of: injection, oral, pulmonary, nasal, and transdermal.

9. The method of claim 1, wherein leptin is administered by inhalation.

10. The method of claim 1, wherein administration of leptin results in serum or plasma leptin levels of at least 25 ng/ml.

11. The method of claim 10, wherein administration of leptin results in serum or plasma leptin levels of from 25 to 100 ng/ml.

12. The method of claim 10, wherein administration of leptin results in serum or plasma leptin levels of from 20 to 200 ng/ml.

13. The method of claim 1, further comprising the step of measuring serum or plasma leptin levels prior to administration of leptin.

14. The method of claim 1, wherein the leptin is attached to polyethylene glycol.
